(19) United States
(12) Patent Application Publication
(10) Pub. No.: US 2005/0101565 A1
(43) Pub. Date: May 12, 2005

(54) Pharmaceutical compositions and methods for treating, preventing, and managing cholesterol, dyslipidemia, and related disorders

(75) Inventor: Jean-Louis Henry Dasseux, Brighton, MI (US)

Correspondence Address: JONES DAY, 51 Louisiana Aveue, N.W, WASHINGTON, DC, 20001-2113, US

(73) Assignee: Esperion Therapeutics, Inc.

(21) Appl. No.: 10/610,682

(22) Filed: Jul. 2, 2003

Related U.S. Application Data
(60) Provisional application No. 60/393,184, filed on Jul. 3, 2002.

(51) Int. Cl. A61K 31/724; A61K 31/4439; A61K 31/426; A61K 31/40; A61K 31/19; A61K 31/175; A61K 31/155

(52) U.S. Cl. 514/58; 514/423; 514/460; 514/592; 514/571; 514/369; 514/342; 514/635

(57) ABSTRACT

The invention relates, in part, to pharmaceutical compositions that comprise a combination of pantethine, or a derivative thereof, and a second active agent. Examples of second active agents include, but are not limited to, statins, fibrates, glitazones, biguanides, sulfonylureas, dyslipidemic controlling compounds, small peptides of the invention, and combinations thereof. The invention also relates to methods for treating, preventing, or managing cholesterol, dyslipidemia, and related disorders.

This application claims the benefit of U.S. provisional application No. 60/393,184, filed Jul. 3, 2002, the disclosure of which is incorporated herein in its entirety.

### 1. FIELD OF THE INVENTION

The invention relates, in part, to pharmaceutical compositions that comprise a combination of pantethine, or a derivative thereof, and a second active agent. The invention also relates to methods for treating, preventing, or managing cholesterol, dyslipidemia, and related disorders.

### 2. BACKGROUND OF THE INVENTION

Dyslipidemia is caused by various factors including, but not limited to, high total cholesterol, high triglycerides, low high-density lipoprotein cholesterol, normal to elevated low-density lipoprotein cholesterol, or small low-density lipoprotein particles. These factors are also related to various other disorders.

The evidence linking elevated serum cholesterol to coronary heart disease is overwhelming. (Badimon et al., Circulation, 86 Suppl. III, 1992, 86-94). Circulating cholesterol is carried by plasma lipoproteins, which are complex particles of lipid and protein that transport lipids in the blood. Low density lipoprotein (LDL) and high density lipoprotein (HDL) are the major cholesterol-carrier proteins. Id. LDL is believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. Id. HDL is also responsible for the removal non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, IDL and LDL. See Badimon et al., 1992, Circulation 86:(Suppl. III)86-94. The apo B-containing lipoprotein, and in particular LDL, has popularly become known as the “bad” cholesterol. In contrast, HDL serum levels correlate inversely with coronary heart disease. Indeed, high serum levels of HDL is regarded as a negative risk factor. It is hypothesized that high levels of plasma HDL is not only protective against coronary artery disease, but may actually induce regression of atherosclerotic plaque. See Dansky and Fisher, 1999, Circulation 100: 1762-3. Thus, HDL has popularly become known as the “good” cholesterol.

### 2.1 Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the bloodstream. The core of VLDL consists mostly of triglycerides synthesized in the liver, with a smaller amount of cholesteryl esters either synthesized in the liver or recycled from chylomicrons. Jacob et al., Journal of Nutrition, 1999; volume 129: pages 712-717. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When a VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins. Id.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

The liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. In addition, remnant receptors are responsible for clearing chylomicrons and VLDL remnants (i.e., IDL). However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL, the “bad” cholesterol, are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery and causing a heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the cell's ability to make its own cholesterol by turning off the synthesis of HMG-CoA reductase, a key enzyme in the cholesterol biosynthetic pathway. Second, the incoming LDL-derived cholesterol promotes storage of cholesterol by the action of ACAT, the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading (For a review, See Mahley & Bersot, The Pharmacological Basis Of Therapeutics, 10th Ed., Goodman & Gilman, Pergaman Press, NY, 2001, Ch. 36, pp. 971-1002).

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT. LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarcation, stroke, cerebral infarction, and restenosis following angioplasty.

### 2.2 Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and circulates in plasma associated with the HDL fraction. Cholesterol ester transfer protein (CETP) and another lipid transfer protein, phospholipid transfer protein (PLTP), contribute to further remodeling the circulating HDL population. See Bruce et al., 1998, Annu. Rev. Nutr. 18:297-330. PLTP supplies lecithin to HDL, and CETP can move cholesteryl ester made by LCAT to other lipoproteins, particularly apoB-containing lipoproteins, such as VLDL. HDL triglyceride can be catabolized by the extracellular hepatic triglyceride lipase, and lipoprotein cholesterol is removed by the liver via several mechanisms.

Each HDL particle contains at least one molecule, and usually two to four molecules, of apolipoprotein (apo A-I). Apo A-I is synthesized by the liver and small intestine as preproapolipoprotein which is secreted as a proprotein that is rapidly cleaved to generate a mature polypeptide having 243 amino acid residues. Apo A-I consists mainly of a 22 amino acid repeating segment, spaced with helix-breaking proline residues. Apo A-I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL₃ and HDL₂). Most HDL in the circulating population contains both apo A-I and apo A-II, a second major HDL protein. This apo A-I- and apo A-II-containing fraction is referred to herein as the AI/AII-HDL fraction of HDL. But the fraction of HDL containing only apo A-I, referred to herein as the AI-HDL fraction, appears to be more effective in RCT. Certain epidemiologic studies support the hypothesis that the AI-HDL fraction is antiartherogenic (Parra et al., 1992, Arterioscler. Thromb. 12:701-707; Decossin et al., 1997, Eur. J. Clin. Invest. 27:299-307).

Although the mechanism for cholesterol transfer from the cell surface is unknown, it is believed that the lipid-poor complex, pre-beta-1 HDL, is the preferred acceptor for cholesterol transferred from peripheral tissue involved in RCT. Cholesterol newly transferred to pre-beta-1 HDL from the cell surface rapidly appears in the discoidal pre-beta-2 HDL. PLTP may increase the rate of disc formation (Lagrost et al., 1996, J. Biol. Chem. 271:19058-19065), but data indicating a role for PLTP in RCT is lacking. LCAT reacts preferentially with discoidal and spherical HDL, transferring the 2-acyl group of lecithin or phosphatidylethanolamine to the free hydroxyl residue of fatty alcohols, particularly cholesterol, to generate cholesteryl esters (retained in the HDL) and lysolecithin. The LCAT reaction requires an apoliprotein such apo A-I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol. Cholesteryl esters in the mature HDL particles of the AI-HDL fraction are removed by the liver and processed into bile more effectively than those derived from the AI/AII-HDL fraction. This may be due, in part, to the more effective binding of AI-HDL to the hepatocyte membrane. Several HDL receptor receptors have been identified, the most well characterized of which is the scavenger receptor class B, type I (SR-BI) (Acton et al., 1996, Science 271:518-520). The SR-BI is expressed most abundantly in steroidogenic tissues (e.g., the adrenals), and in the liver (Landshulz et al., 1996, J. Clin. Invest. 98:984-995; Rigotti et al., 1996, J. Biol. Chem. 271:33545-33549). Other proposed HDL receptors include HB1 and HB2 (Hidaka and Fidge, 1992, Biochem J. 15:161-7; Kurata et al., 1998, J. Atherosclerosis and Thrombosis 4:112-7).

While there is a consensus that CETP is involved in the metabolism of VLDL- and LDL-derived lipids, its role in RCT remains controversial. However, changes in CETP activity or its acceptors, VLDL and LDL, play a role in “remodeling” the HDL population. For example, in the absence of CETP, the HDL becomes enlarged particles that are poorly removed from the circulation (for reviews on RCT and HDLs, see Fielding & Fielding, 1995, J. Lipid Res. 36:211-228; Barrans et al., 1996, Biochem. Biophys. Acta. 1300:73-85; Hirano et al., 1997, Arterioscler. Thromb. Vasc. Biol. 17:1053-1059).

### 2.3 Reverse Transport of Other Lipids

HDL is not only involved in the reverse transport of cholesterol, but also plays a role in the reverse transport of other lipids, i.e., the transport of lipids from cells, organs, and tissues to the liver for catabolism and excretion. Such lipids include sphingomyelin, oxidized lipids, and lysophophatidylcholine. For example, Robins and Fasulo (1997, J. Clin. Invest. 99:380-384) have shown that HDL stimulates the transport of plant sterol by the liver into bile secretions.

### 2.4 Current Cholesterol Management Therapies

In the past two decades or so, the segregation of cholesterolemic compounds into HDL and LDL regulators and recognition of the desirability of decreasing blood levels of the latter have led to the development of a number of drugs. Fisher et al., J. Biol Chem. 1985, 260:15745-15751. However, many of these drugs have undesirable side effects and/or are contraindicated in certain patients, particularly when administered in combination with other drugs.

One compound that reportedly can reduce blood levels of triglycerides and cholesterol when administered to a patient is pantethine. Pantethine, which is chemically named D-bis-(N-pantothenyl-beta-aminoethyl)-disulfide, has the following structure:

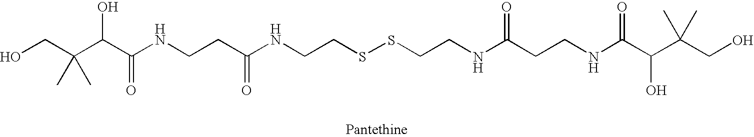

Pantethine is a disulfide form of pantothenic acid or vitamin B5 with a cysteine moiety. The body uses pantothenic acid to make proteins as well as other important chemicals needed to metabolize fats and carbohydrates. For example, some studies have suggested that pantothenic acid is a direct precursor to Coenzyme A (CoA). Alt. Med. Rev. 1997, 2(5):365-377. CoA, one of the most important substances in body metabolism, participates in the following fundamental metabolic functions: the synthesis of fatty acids, the degradation of fatty acids, the Krebs cycle in which most of the body's energy is produced, the acetylation of choline-the major neurotransmitter of the body, the synthesis of antibodies, the utilization of nutrients-including fats, proteins, and carbohydrates, the maintenance of blood sugar levels, the synthesis of porphyrin-a heme precursor of importance in hemoglobin synthesis, the metabolism of some minerals and trace elements, the synthesis of steroid hormones, and the detoxification of drugs, including sulphonamides. Pantothenic acid is also used in the manufacture of hormones, red blood cells, and acetylcholine, an important neurotransmitter (signal carrier between nerve cells). As a supplement, pantothenic acid has been proposed as a treatment for rheumatoid arthritis, an athletic performance enhancer, and an “antistress” nutrient.

Both animal and human studies have shown that the administration of pantethine can help those with hyperlipidemias including hypercholesterolemia. See Arsenio et al., Clinical Therapeutics, Vol. 8, No. 5, 1986, pp. 537-545; see also Tawara et al., Japan J. Pharmacol., 41, 1986, 211-222; Cighetti et al., Artheroscelerosis, 60, 1986, 67-77; & Da Col et al., Therapeutic Research, Vol. 36, No. 2, 1984, 314-322, each of which is incorporated herein by reference. Pantethine has been shown to reduce tissue levels of acetaldehyde and as such may be an useful adjunct in protocols for chronic candidiasis and alcoholism. Research has also indicated that the activity of aldehyde dehydrogenase may be increased by supplementation with pantethine. This enzyme is responsible for the breakdown of formaldehyde and as such pantethine may be helpful to those with formaldehyde sensitivity. Other research indicates that pantethine depletes cystine from cystinotic fibroblasts and as such may be effective in treating cystinosis.

Other compounds that are reportedly useful in the treatment of cholesterol disorders are bile-acid-binding resins, which are a class of drugs that interrupt the recycling of bile acids from the intestine to the liver. Examples of bile-acid-binding resins, include, but are not limited to, cholestyramine (QLESTRAN LIGHT®, Bristol-Myers Squibb), and colestipol hydrochloride (COLESTID®, Pharmacia & Upjohn Company). When taken orally, these positively charged resins bind to negatively charged bile acids in the intestine. Because the resins cannot be absorbed from the intestine, they are excreted, carrying the bile acids with them. The use of such resins, however, at best only lowers serum cholesterol levels by about 20%. Moreover, their use is associated with gastrointestinal side-effects, including constipation and certain vitamin deficiencies. Moreover, since the resins bind to drugs, other oral medications must be taken at least one hour before or four to six hours subsequent to ingestion of the resin, complicating heart patients' drug regimens.

The statins are inhibitors of cholesterol synthesis. Lovastatin (MEVACOR®, Merck & Co., Inc.), a natural product derived from a strain of Aspergillus; pravastatin (PRAVACHOL®, Bristol-Myers Squibb Co.); fluvastatin (LESCOL®&, Reliant), rosuvastatin (CRESTOR®, Astra-Zeneca), and atorvastatin (LIPITOR®, Warner Lambert) block cholesterol synthesis by inhibiting HMGCoA, the key enzyme involved in the cholesterol biosynthetic pathway. Lovastatin significantly reduces serum cholesterol and LDL-serum levels. It also slows progression of coronary atherosclerosis. However, serum HDL levels are only slightly increased following lovastatin administration. The mechanism of the LDL-lowering effect may involve both reduction of VLDL concentration and induction of cellular expression of LDL-receptor, leading to reduced production and/or increased catabolism of LDL. Side effects, including liver and kidney dysfunction are associated with the use of these drugs. International Applications, WO 02/47682 and 02/47683, disclose “blood lipid ameliorant compositions,” which comprises simvastatin (ZOCOR®, Merck & Co. Inc.) or atorvastatin (LIPITOR®, Parke-Davis), respectively, in combination with one or more compounds, one of which is pantethine.

Niacin, also known as nicotinic acid, is a water-soluble vitamin B-complex used as a dietary supplement and antihyperlipidemic agent. Niacin diminishes production of VLDL and is effective at lowering LDL. NIASPAN® has been shown to increase HDL when administered at therapeutically effective doses; however, its usefulness is limited by serious side effects.

Fibrates are a class of lipid-lowering drugs used to treat various forms of hyperlipidemia or elevated serum triglycerides, which may also be associated with hypercholesterolemia. Fibrates appear to reduce the VLDL fraction and modestly increase HDL; however, the effects of these drugs on serum cholesterol is variable. In the United States, fibrates have been approved for use as antilipidemic drugs, but have not received approval as hypercholesterolemia agents. For example, clofibrate (ATROMID-S®, Wyeth-Ayerst Laboratories) is an antilipidemic agent that acts to lower serum triglycerides by reducing the VLDL fraction. Although ATROMID-S may reduce serum cholesterol levels in certain patient subpopulations, the biochemical response to the drug is variable, and is not always possible to predict which patients will obtain favorable results. ATROMID-S has not been shown to be effective for prevention of coronary heart disease. The chemically and pharmacologically related drug, gemfibrozil (LOPID, Parke-Davis), is a lipid regulating agent which moderately decreases serum triglycerides and VLDL cholesterol. LOPID also increases HDL cholesterol, particularly the HDL₂ and HDL₃ subfractions, as well as both the AI/AII-HDL fraction. However, the lipid response to LOPID is heterogeneous, especially among different patient populations. Fenofibrate (TRICOR®, Abbott) may reduce triglyceride levels as well as VLDL levels in blood. Moreover, while prevention of coronary heart disease was observed in male patients between the ages of 40 and 55 without history or symptoms of existing coronary heart disease, it is not clear to what extent these findings can be extrapolated to other patient populations (e.g., women, older and younger males). Indeed, no efficacy was observed in patients with established coronary heart disease. Serious side-effects are associated with the use of fibrates, including toxicity; malignancy, particularly malignancy of gastrointestinal cancer; gallbladder disease; and an increased incidence in non-coronary mortality. These drugs are not indicated for the treatment of patients with high LDL or low HDL as their only lipid abnormality.

Biguanides for use in combination with the compounds of the invention include but are not limited to metformin, phenformin and buformin. Metformin is a biguanide that has been used worldwide for the treatment of type 2 diabetes for the past 4 decades. It improves glycemic control by enhancing insulin sensitivity in the liver and in muscle. Improved metabolic control with metformin does not induce weight gain and may cause weight loss.

Metformin also has a beneficial effect on several cardiovascular risk factors including dyslipidemia, elevated plasminogen activator inhibitor 1 levels, other fibrinolytic abnormalities, hyperinsulinemia, and insulin resistance. While metformin reduces insulin resistance, the cellular mechanism of action is incompletely understood. Metformin enhances muscle and adipocyte insulin receptor number and/or affinity, increases insulin receptor tyrosine kinase activity, stimulates glucose transport and glycogen synthesis, and reduces both hepatic gluconeogenesis and glycogenolysis. In addition, metformin has been reported to decrease lipid oxidation and plasma free fatty acid levels, leading to an inhibition of an overactive Randle cycle.

Side effects of metformin are primarily confined to the gastrointestinal tract (abdominal discomfort and diarrhea). These side effects can be minimized by slow titration and administration with food. Lactic acidosis is rare, with an incidence of 3 cases per 100,000 patient/year of therapy. Most reported cases of lactic acidosis occur in patients with contraindications, particularly impaired renal function (>90% of cases). Metformin is an effective and safe therapeutic agent for the treatment of type 2 diabetes. Its ability to improve insulin sensitivity and the cardiovascular risk profile of type 2 diabetic patients has enhanced its clinical use as first-line therapy. Metformin also reduced diabetes-related death, heart attacks, and stroke.

Metformin is indicated for patients with non-insulin-dependent diabetes mellitus (NIDDM), particularly those with refractory obesity. It is used either as monotherapy (with dietary measures) or in combination with a sulfonylurea product such as glyburide. Also, there is suggestive evidence that insulin requirement for type I diabetic patients may be reduced with the combined long-term use of metformin and insulin. Based on laboratory and clinical studies and without being limited by theory, several mechanisms of action have been proposed: enhanced peripheral glucose uptake and utilization; inhibition of hepatic gluconeogenesis (glucose production); increased muscle glyconeogenesis (production of new glycogen molecules); reduction of net glucose absorption by the small intestine; reduction of plasma glucagon level; and increased insulin receptor affinity (reduced insulin resistance).

The inhibition of hepatic glucose production is reportedly the most significant pharmacological action of metformin. In contrast to the sulfonyulureas, whose primary mechanism of action is to increase the release of endogenous insulin, the biguanides apparently have no effect on the function of the cells of the pancreas. Also, metformin has no significant hypoglycemic effect in fasting, non-diabetic individuals, and it does not cause hypoglycemia in diabetic patients (except perhaps in combination with physical exercise). Many patients experience a modest but significant weight reduction (5-10%) with metformin therapy.

Sulfonylureas are indicated to treat type 2 diabetes when a nutrition and exercise program alone fails to control blood sugar. Sulfonylureas may be used as the only diabetes medicine, taken with another diabetes pill, or taken with insulin injections. The medicines have been in use for the past 40 years. Sulfonylureas reportedly lower blood sugar by helping the pancreas produce more insulin and making the muscles and liver use up excess sugar. Glipizide (GLUCOTROL®, Pfizer) is a sulfonylurea drug used to lower blood sugar levels in people with non-insulin-dependent diabetes mellitus. Sulfonylureas lower blood sugar only if the body produces some insulin. Side effects associated with sulfonylureas include low blood sugar (hypoglycemia), weight gain, and allergic reactions in people with an allergy to sulfa-medicines.

Hypertension or high blood pressure adds to the workload of the heart and arteries. If it continues for a long time, the heart and arteries may not function properly. This can damage the blood vessels of the brain, heart, and kidneys, resulting in a stroke, heart failure, or kidney failure. High blood pressure may also increase the risk of heart attacks. These problems may be less likely to occur if blood pressure is controlled by an antihypertensive drug, such as, for example, b-blockers, acetylcholinesterase (ACE) inhibitors, or angiotensin II receptor blockers. Losartan (COZAAR®, Merck and Co. Inc.) is another drug used to treat high blood pressure. Losartan works by blocking the action of a substance in the body that causes blood vessels to tighten. As a result, losartan relaxes blood vessels. This lowers blood pressure and increases the supply of blood and oxygen to the heart.

Oral estrogen replacement therapy may be considered for moderate hypercholesterolemia in post-menopausal women. However, increases in HDL may be accompanied with an increase in triglycerides. Estrogen treatment is, of course, limited to a specific patient population, postmenopausal women, and is associated with serious side effects, including induction of malignant neoplasms; gall bladder disease; thromboembolic disease; hepatic adenoma; elevated blood pressure; glucose intolerance; and hypercalcemia.

Long chain carboxylic acids, particularly long chain α,ω-dicarboxylic acids with distinctive substitution patterns, and their simple derivatives and salts, have been disclosed for treating atherosclerosis, obesity, and diabetes (see, e.g., Bisgaier et al., 1998, J. Lipid Res. 39:17-30, and references cited therein; International Patent Publication WO 98/30530; U.S. Pat. No. 4,689,344; International Patent Publication WO 99/00116; and U.S. Pat. No. 5,756,344). However, some of these compounds, for example the α,ω-dicarboxylic acids substituted at their α,α′-carbons (U.S. Pat. No. 3,773,946), while having serum triglyceride and serum cholesterol-lowering activities, reportedly have no value for treatment of obesity and hypercholesterolemia (U.S. Pat. No. 4,689,344).

U.S. Pat. No. 4,689,344 discloses β,β,β′,β′-tetrasubstituted-α,ω-alkanedioic acids that are optionally substituted at their α,α,α′,α′ positions, and alleges that they are useful for treating obesity, hyperlipidemia, and diabetes. According to this reference, both triglycerides and cholesterol are lowered significantly by compounds such as 3,3,14,14-tetramethylhexadecane-1,16-dioic acid. U.S. Pat. No. 4,689,344 further reports that the β,β,β′,β′-tetramethyl-alkanediols of U.S. Pat. No. 3,930,024 also are not useful for treating hypercholesterolemia or obesity.

Other compounds that reportedly have activity in treating cholesterol disorders are disclosed in U.S. Pat. No. 4,711,896. In U.S. Pat. No. 5,756,544, α,ω-dicarboxylic acid-terminated dialkane ethers are disclosed to have activity in lowering certain plasma lipids, including Lp(a), triglycerides, VLDL-cholesterol, and LDL-cholesterol, in animals, and elevating others, such as HDL-cholesterol. The compounds are also stated to increase insulin sensitivity. In U.S. Pat. No. 4,613,593, phosphates of dolichol, a polyprenol isolated from swine liver, are stated to be useful in regenerating liver tissue, and in treating hyperuricuria, hyperlipemia, diabetes, and hepatic diseases in general.

U.S. Pat. No. 4,287,200 discloses azolidinedione derivatives with anti-diabetic, hypolipidemic, and anti-hypertensive properties. However, the administration of these compounds to patients can produce side effects such as bone marrow depression, and both liver and cardiac cytotoxicity. The compounds disclosed in U.S. Pat. No. 4,287,200 reportedly stimulate weight gain in obese patients.

Peroxisome proliferators are a structurally diverse group of compounds that, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, 1985, Ann. Rev. Cell Biol. 1:489-530; Vamecq and Draye, 1989, Essays Biochem. 24:1115-225; and Nelali et al., 1988, Cancer Res. 48:5316-5324). Chemicals included in this group are the fibrate class of hypolipidermic drugs, herbicides, and phthalate plasticizers (Reddy and Lalwani, 1983, Crit. Rev. Toxicol. 12:1-58). Peroxisome proliferation can also be elicited by dietary or physiological factors, such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, 1990, Nature 347:645-650). This receptor, termed peroxisome proliferator activated receptor α (PPAR_{α}), was subsequently shown to be activated by a variety of medium and long-chain fatty acids. PPAR_{α} activates transcription by binding to DNA sequence elements, termed peroxisome proliferator response elements (PPRE), in the form of a heterodimer with the retinoid X receptor (RXR). RXR is activated by 9-cis retinoic acid (see Kliewer et al., 1992, Nature 358:771-774; Gearing et al., 1993, Proc. Natl. Acad. Sci. USA 90:1440-1444, Keller et al., 1993, Proc. Natl. Acad. Sci. USA 90:2160-2164; Heyman et al., 1992, Cell 68:397-406, and Levin et al., 1992, Nature 355:359-361). Since the discovery of PPAP_{α}, additional isoforms of PPAR have been identified, e.g., PPAR_{β}, PPAR_{γ} and PPAR_{δ}, which are have similar functions and are similarly regulated.

PPREs have been identified in the enhancers of a number of genes encoding proteins that regulate lipid metabolism. These proteins include the three enzymes required for peroxisomal β-oxidation of fatty acids; apolipoprotein A-I; medium-chain acyl-CoA dehydrogenase, a key enzyme in mitochondrial β-oxidation; and aP2, a lipid binding protein expressed exclusively in adipocytes (reviewed in Keller and Whali, 1993, TEM, 4:291-296; see also Staels and Auwerx, 1998, Atherosclerosis 137 Suppl:S19-23). The nature of the PPAR target genes coupled with the activation of PPARs by fatty acids and hypolipidemic drugs suggests a physiological role for the PPARs in lipid homeostasis.

Pioglitazone, an antidiabetic compound of the thiazolidinedione class, was reported to stimulate expression of a chimeric gene containing the enhancer/promoter of the lipid-binding protein aP2 upstream of the chloroamphenicol acetyl transferase reporter gene (Harris and Kletzien, 1994, Mol. Pharmacol. 45:439-445). Deletion analysis led to the identification of an approximately 30 bp region responsible for pioglitazone responsiveness. In an independent study, this 30 bp fragment was shown to contain a PPRE (Tontonoz et al., 1994, Nucleic Acids Res. 22:5628-5634). Taken together, these studies suggested the possibility that the thiazolidinediones modulate gene expression at the transcriptional level through interactions with a PPAR and reinforce the concept of the interrelatedness of glucose and lipid metabolism.

Despite the reported advantages of various drugs used for cholesterol management, a need still exists for pharmaceutical compositions and therapies that can be used to regulate dyslipidemia, lipoprotein, insulin and/or glucose levels in the blood. Further, a need exists for safer and more efficacious methods of lowering serum cholesterol, increasing HDL serum levels, preventing coronary heart disease, and/or treating existing diseases such as, but not limited to, atherosclerosis, obesity, diabetes, and other diseases that are affected by lipid metabolism and/or lipid levels. There is also is a need for pharmaceutical compositions that may be used with other lipid-altering treatment regimens in a synergistic manner.

### 3. SUMMARY OF THE INVENTION

The invention encompasses pharmaceutical compositions comprising pantethine or a derivative thereof and a second active agent or a derivative thereof. Second active agents include, but are not limited to, statins, fibrates, bigaunides, glitazones, sulfonylureas, small dyslipidemic controlling compounds, small peptides of the invention, and combinations thereof.

Pharmaceutical compositions of the invention are useful for treating, preventing, or managing cholesterol, dyslipidemia, and related disorders including, but not limited to: cardiovascular disease; artherosclerosis; stroke; peripheral vascular disease; dyslipidemia; dyslipoproteinemia; restenosis; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; impotence; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus (NIDDM); septic shock; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease.

The invention also encompasses methods of treating, preventing, or managing a cholesterol, dyslipidemia, or related disorder that comprise administering to a patient in need of such treatment, prevention, or management an effective amount of pantethine or a derivative thereof and a second active agent or a derivative thereof.

The invention also encompasses methods of treating, preventing, or managing a cholesterol, dyslipidemia, or related disorder that comprise administering for at least thirty days to a patient in need of such treatment, prevention, or management an effective amount of pantethine or a derivative thereof and a second active agent or a derivative thereof.

The invention further encompasses methods of reducing or avoiding an adverse effect associated with pantethine monotherapy, which comprise administering to a patient in need thereof an effective amount of a combination of pantethine and a second active agent.

The invention further encompasses methods of reducing or avoiding an adverse effect associated with second active agent monotherapy, which comprise administering to a patient in need thereof an effective amount of a combination of pantethine and a second active agent.

### 3.1 Definitions

As used herein and unless otherwise indicated, the term “patient” means animal such as a mammal or bird. Examples of a patient include, but is not limited to, a cow, monkey, horse, sheep, pig, chicken, turkey, quail, cat, dog, mouse, rat, rabbit, guinea pig, or human. Preferred patients are human.

As used herein and unless otherwise indicated, the term “halogen” or “halo” means —F, —Cl, —Br, or —I.

As used herein and unless otherwise indicated, the term “alkyl” means a saturated straight chain or branched non-cyclic hydrocarbon having from 1 to 10 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein and unless otherwise indicated, the term “alkenyl” means a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, 2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

As used herein and unless otherwise indicated, the term “alkynyl” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

As used herein and unless otherwise indicated, the term “phenyl” means —C₆H₅. A phenyl group can be unsubstituted or substituted with one or two suitable substituents.

As used herein and unless otherwise indicated, the term “benzyl” means —CH₂-phenyl.

As used herein and unless otherwise indicated, the term “composition of the invention” refers to a composition comprising panthethine or a derivative thereof, and a second active agent.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition or compound. Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

As used herein and unless otherwise indicated, the term “second active agent” refers to a compound or mixture of compounds that are combined and/or administered with pantethine, or a derivative thereof, according to the invention. Examples of second active agents include, but are not limited to, statins, fibrates, glitazones, biguanides, dyslipidemic controlling compounds, small peptides of the invention, and pharmaceutically acceptable salts, solvates, prodrugs thereof, and combinations thereof.

As used herein and unless otherwise indicated, the term “third active agent” refers to a compound or mixture of compounds that are combined and/or administered with pantethine, or a derivative thereof, and a second active agent. Specific third active agents reduce a disorder such as, but not limited to, hepatotoxicity, myopathy, cataracts, or rhabdomyolysis. Examples of third active agents include, but not limited to, bile acid-binding resins; niacin; hormones and pharmaceutically acceptable salts, solvates, prodrugs thereof, and combinations thereof.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the term “pharmaceutically acceptable salt(s),” includes, but is not limited to, salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate,” means a compound of the invention or a salt thereof, that further includes a stoichiometric or non-stoichiometric amount of a solvent bound by non-covalent intermolecular forces. Preferred solvents are volatile, non-toxic, and/or acceptable for administration to humans in trace amounts. The term solvate includes hydrates and means a compound of the invention or a salt thereof, that further includes a stoichiometric or non-stoichiometric amount of water bound by non-covalent intermolecular forces and includes a mono-hydrate, dihydrate, trihydrate, tetrahydrate, and the like.

As used herein and unless otherwise indicated, the term “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, and —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “monotherapy” means the administration of a single drug not in conjunction with any other drugs.

As used herein and unless otherwise indicated, the term “substituted” as used to describe a compound or chemical moiety means that at least one hydrogen atom of that compound or chemical moiety is replaced with a second chemical moiety. Examples of second chemical moieties include, but are not limited to: halogen atoms (e.g., chlorine, bromine, and iodine); C₁-C₆ linear, branched, or cyclic alkyl (e.g., methyl, ethyl, butyl, tert-butyl, and cyclobutyl); hydroxyl; thiols; carboxylic acids; esters, amides, silanes, nitriles, thioethers, stannanes, and primary, secondary, and tertiary amines (e.g., —NH₂, —NH(CH₃), —N(CH₃)₂, and cyclic amines). Preferred second chemical moieties are chlorine, hydroxyl, methoxy, amine, thiol, and carboxylic acid.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### 4. DETAILED DESCRIPTION OF THE INVENTION

This invention is based, in part, on the belief that pantethine or a derivative thereof and a second active agent can be used in the treatment, prevention, or management of cholesterol, dyslipidemia, and related disorders. Without being limited by theory, it is believed that pantethine and derivatives thereof may act in complementary or synergistic ways with certain other compounds when used to treat, prevent, or manage cholesterol, dyslipidemia, or related disorders. It is also believed that pantethine or a derivative thereof may be used to reduce or eliminate particular adverse effects associated with certain drugs (e.g., second active agents). It is further believed that certain drugs (e.g., second active agents), may be used to reduce or eliminate particular adverse effects associated with pantethine monotherapy. It is also believed that pantethine, or a derivative thereof may be used to reverse adverse effects associated with certain drugs (e.g., second active agents).

A first embodiment of the invention encompasses pharmaceutical compositions comprising pantethine, or a derivative thereof and a second active agent. Specific second active agents include, but are not limited to, statins, fibrates, biguanides, glitazones, sulfonylureas, dyslipidemic compounds of the invention, peptides of the invention, and combinations thereof.

In another embodiment, the pharmaceutical compositions of the invention farther comprise a third active agent. Examples of a third active agent include, but are not limited to, bile acid-binding resins, niacin, hormones, or pharmaceutically acceptable salts, solvates, clathrates, polymorphs, prodrugs, and combinations thereof.

Another embodiment of the invention encompasses a method of treating, preventing, or managing dyslipidemia or a cholesterol disorder, which comprises administering to a patient in need of such treatment, prevention, or management an effective amount of pantethine, or a derivative thereof, and a second active agent. Examples of dyslipidemia or cholesterol disorder include, but are not limited to: cardiovascular disease, stroke, and peripheral vascular disease; dyslipidemia; dyslipoproteinemia; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease.

Another embodiment of the invention encompasses a method of treating, preventing, or managing dyslipidemia or a cholesterol disorder, which comprises administering for at least thirty days to a patient in need of such treatment, prevention, or management an effective amount of pantethine, or a derivative thereof, and a second active agent, wherein the second active agent is a statin, fibrate, glitazone, biguanide, sulfonylurea, a dyslipidemic controlling compound, a peptide of the invention, or a pharmaceutically acceptable salt, solvate, clathrate, polymorph, prodrug, or pharmacologically active metabolite thereof.

Another embodiment of the invention encompasses a method of reducing or avoiding an adverse effect associated with pantethine monotherapy, which comprises administering to a patient in need thereof an effective amount of a combination of pantethine and a second active agent, wherein the second active agent is a statin, fibrate, glitazone, biguanide, sulfonylurea, a dyslipidemic controlling compound, a peptide of the invention, or a pharmaceutically acceptable salt, solvate, clathrate, polymorph, prodrug, or pharmacologically active metabolite thereof.

Another embodiment of the invention encompasses a method of reducing or avoiding an adverse effect associated with second active agent monotherapy, which comprises administering to a patient in need thereof an effective amount of a combination of pantethine and a second active agent, wherein the second active agent is a statin, fibrate, glitazone, biguanide, sulfonylurea, a dyslipidemic controlling compound, small peptide of the invention, or a pharmaceutically acceptable salt, solvate, clathrate, polymorph, prodrug, or pharmacologically active metabolite thereof.

Specific adverse effects include, but are not limited to, hepatotoxicity, myopathy, cataracts, rhabdomyolysis, life threatening ventricular arrhythmia, heart failure, atrial fibrillation, atrial flutter, venous congestion, edema, dyspnea, orthopnea, cardiac asthma, palpitation, hypertension, hypotension, and precordial distress or weakness.

### 4.1 Pantethine and Derivatives Thereof

Pantethine is the disulfide form of pantetheine. Pantetheine, which is chemically named 2,4-Dihydroxy-N-[2-(2-mercapto-ethylcarbamoyl)-ethyl]-3,3-dimethyl-butyramide, can exist in the following stereoisomeric forms:

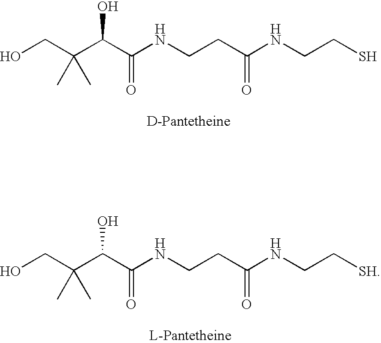

Pantethine, the dimer of pantetheine, can thus exist in the following stereoisomeric forms:

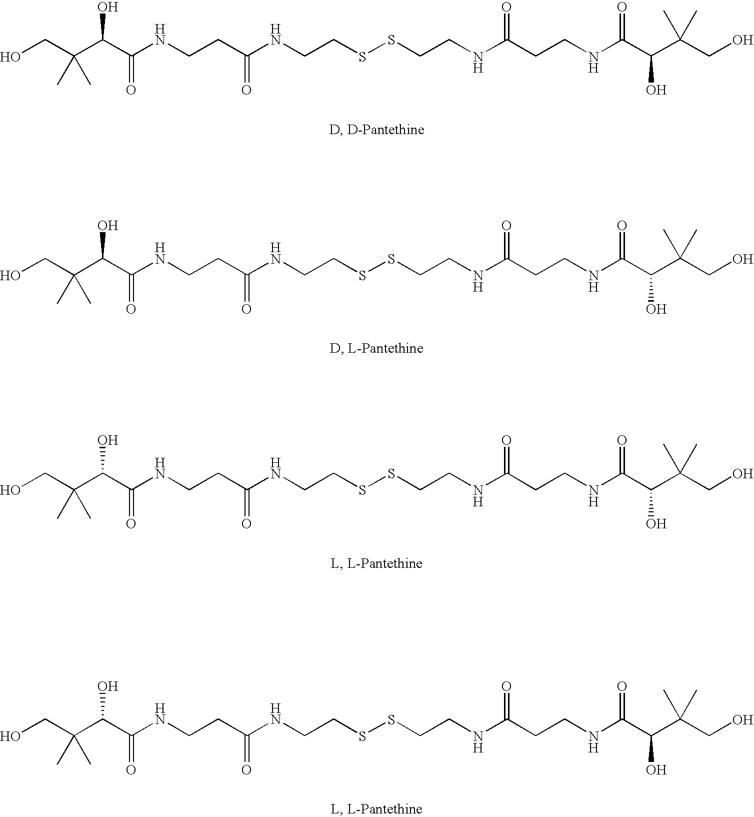

Pantothenic acid, which is also known as vitamin B5, can exist in the following two stereoisomeric forms:

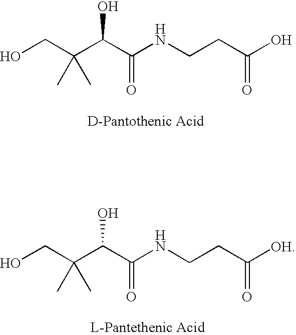

Phospho-pantetheine can exist in the following stereoisomeric forms:

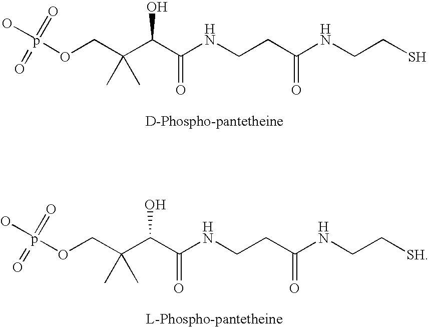

As used herein and unless otherwise indicated, the phrases “pantethine or a derivative thereof,” and “pantethine or derivatives thereof,” encompass, but are not limited to, D,D-pantethine, D,L-pantethine, L,L-pantethine, L,D-pantethine, D-pantetheine, L-pantetheine, D-phospho-pantetheine, L-phospho-pantetheine, D-pantothenic acid, L-pantothenic acid, mixtures thereof, or a pharmaceutically acceptable salt, solvate, clathrate, polymorph, prodrug, or pharmacologically active metabolite thereof.

In one embodiment of the invention, the pantethine or a derivative thereof is stereomerically pure. In another embodiment of the invention, the pantethine or a derivative thereof is a diastereomeric or racemic mixture. In a particular embodiment of the invention, the term “pantethine or a derivative thereof” does not encompass D,D-pantethine.

### 4.2 Second Active Agents

It is believed that a second active agent can be used in combination with pantethine or a derivative thereof, for use in the treatment, prevention, or management of cholesterol, dyslipidemia, and related disorders. Without being limited by theory, it is believed that second active agents may act in complementary or synergistic ways with pantethine or a derivative thereof, when used to treat, prevent, or manage cholesterol, dyslipidemia, or related disorders.

### 4.2.1 Statins

Statin are drugs that competitively inhibit 3-hydroxy-3-methylglutaryl coenzyme A “HMG-CoA,” which is the enzyme that catalyzes an early, rate limiting step in cholesterol biosynthesis. Hebert et al., JAMA 1997, 278: 313-21. It is believed that the combined use of pantethine, or a derivative thereof, and a statin can typically lower bad “LDL” cholesterol more effectively than other classes of cholesterol-lowering drugs. This combination may also lowers triglyceride, reduce inflammation, and raise protective HDL-cholesterol. It is believed that the combination can have additional therapeutic effects, for example, the combination may lower blood pressure; protect against heart disease, for example, by reducing “smooth muscle proliferation;” reduce heart attacks; reduce platelet aggregation; and to reduce strokes as well as “peripheral arterial disease” (a disease that consists of “clogging” of the arteries to the legs).

Examples of statins of the invention include, but are not limited to, mevastatin, pitavastatin, rosuvastatin, pentostatin (Nipent®), nystatin, lovastatin (Mevacor®), simvastatin (Zocor®), pravastatin (Pravachol®), fluvastatin (Lescol®), atorvastatin (Lipitor®), cerivastatin (Baycol®), combinations thereof, or a pharmaceutically acceptable salt, solvate, clathrate, polymorph, prodrug, or pharmacologically active metabolite thereof. Statins suitable for use in the compositions and methods of the invention are also disclosed in U.S. Pat. Nos. 4,681,893; 5,273,995; 5,356,896; 5,354,772; 5,686,104; 5,969,156; and 6,126,971, each of which is incorporated herein in its entirety by reference. As some statins may exist in an inactive form, such as a lactone (e.g., simvastatin), the invention encompasses using the active form (e.g., b-hydroxy acid form) of them. See Physicians Desk Reference, 54^{th} Ed. (2000) pp. 1917-1920.

Specific methods and pharmaceutical compositions of the invention comprise pantethine or a derivative thereof and mevastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and rosuvastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and pentostatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and nystatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and lovastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and pravastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and fluvastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and pitavastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and cerivastatin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and simvastatin or a pharmaceutically acceptable salt, solvate, clathrate, polymorph, prodrug, or pharmacologically active metabolite thereof. In a particular embodiment of the invention the pantethine or a derivative thereof is not D,D-pantethine.

Other specific methods and pharmaceutical compositions of the invention comprise pantethine or a derivative thereof and atorvastatin or a pharmaceutically acceptable salt, solvate, clathrate, polymorph, prodrug, or pharmacologically active metabolite thereof. In a particular embodiment of the invention the pantethine or a derivative thereof is not D,D-pantethine.

Other specific methods and pharmaceutical compositions of the invention comprise pantethine or a derivative thereof and pharmacologically active metabolite of atorvastatin or a pharmaceutically acceptable salt, solvate, clathrate, polymorph, prodrug thereof.

Other specific methods and pharmaceutical compositions of the invention comprise pantethine or a derivative thereof and pharmacologically active metabolite of simvastatin or a pharmaceutically acceptable salt, solvate, clathrate, polymorph, prodrug thereof.

### 4.2.2 Fibrates

Fibrates or fibric acid derivatives are regarded as broad-spectrum lipid-modulating agents in that although their main action is to decrease serum triglycerides they also tend to reduce LDL-cholesterol and to raise HDL-cholesterol. It is believed that the combined use of pantethine, or a derivative thereof, and a fibrate may reduce the risk of coronary heart disease events in those with low HDL-cholesterol or with raised triglycerides by speeding up the chemical breakdown (i.e., catabolism) of triglyceride-rich lipoproteins that circulate in the body.

Fibrates include, but are not limited to, bezafibrate, ciprofibrate, fenofibrate, gemfibrozil, clofibrate, combinations thereof, or a pharmaceutically acceptable salt, solvate, clathrate, polymorph, prodrug, or pharmacologically active metabolite thereof. Fibrates suitable for inclusion in the compositions or administration in the methods of the invention are disclosed in U.S. Pat. Nos. 4,895,762; 6,074,670; and 6,277,405, each of which is incorporated herein in its entirety. In one embodiment, the methods or compositions of the invention do not include bezafibrate or fenofibrate.

Adverse effects associated with the administration of fibrates include, but are not limited to, a myositis-like syndrome, especially in patients with impaired renal function. Also, the combination of a fibrate with a statin increases the risk of muscle effects (especially rhabdomyolysis) in some patients. Rhabdomyolysis is a rare condition where damage to muscles results in the release of muscle cell contents into the bloodstream, which can lead to serious damage to the kidneys and other organs.

Specific methods and pharmaceutical compositions of the invention comprise pantethine or a derivative thereof and bezafibrate or a pharmaceutically acceptable salt, solvate, clathrate, polymorph, prodrug, or pharmacologically active metabolite thereof.

Other specific methods and pharmaceutical compositions of the invention comprise pantethine or a derivative thereof and ciprofibrate or a pharmaceutically acceptable salt, solvate, clathrate, polymorph, prodrug, or pharmacologically active metabolite thereof.

Other specific methods and pharmaceutical compositions of the invention comprise pantethine or a derivative thereof and fenofibrate or a pharmaceutically acceptable salt, solvate, clathrate, polymorph, prodrug, or pharmacologically active metabolite thereof.

Other specific methods and pharmaceutical compositions of the invention comprise pantethine or a derivative thereof and gemfibrozil or a pharmaceutically acceptable salt, solvate, clathrate, polymorph, prodrug, or pharmacologically active metabolite thereof.

Other specific methods and pharmaceutical compositions of the invention comprise pantethine or a derivative thereof and clofibrate or a pharmaceutically acceptable salt, solvate, clathrate, polymorph, prodrug, or pharmacologically active metabolite thereof.

### 4.2.3 Biguanides

Biguanides for use in the compositions and methods of the invention include, but are not limited to, metformin, phenformin, buformin, combinations thereof, or a pharmaceutically acceptable salt, solvate, clathrate, polymorph, prodrug, or pharmacologically active metabolite thereof. Biguanides suitable for use in the compositions or methods of the invention are also disclosed in U.S. Pat. No. 6,303,146, which is incorporated herein by reference in its entirety.

It is believed that the combined use of pantethine or a derivative thereof and a bigaunide may improve glycemic control by enhancing insulin sensitivity in the liver and in muscle.

It is further believed that the combined use of pantethine or a derivative thereof and a biguanide may reduce or avoid cardiovascular risk factors including, but not limited to, dyslipidemia, elevated plasminogen activator inhibitor 1 levels, other fibrinolytic abnormalities, hyperinsulinemia, insulin resistance, and is an effective and safe therapeutic agent for the treatment of type 2 diabetes.

Specific methods and pharmaceutical compositions of the invention comprise pantethine or a derivative thereof and metformin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and phenformin or a pharmaceutically acceptable salt, solvate, clathrate, polymorph, prodrug, or pharmacologically active metabolite thereof.

Other specific methods and pharmaceutical compositions of the invention comprise pantethine or a derivative thereof and buformin or a pharmaceutically acceptable salt, solvate, clathrate, polymorph, prodrug, or pharmacologically active metabolite thereof.

### 4.2.4 Glitazones

Glitazones include, but are not limited to, 5-((4-(2-(methyl-2-pyridinyl amino)ethoxy)-phenyl)methyl)-2,4-thiazolidinedione, troglitazone, pioglitazone, ciglitazone, WAY-120,744, englitazone, AD 5075, darglitazone, rosiglitazone, combinations thereof, or a pharmaceutically acceptable salt, solvate, clathrate, polymorph, prodrug, or pharmacologically active metabolite thereof. Glitazones suitable for use in the compositions or methods of the invention are disclosed in U.S. Pat. Nos. 4,687,777; 5,002,953; 5,741,803; 5,965,584; 6,150,383; 6,150,384; 6,166,042; 6,166,043; 6,172,090; 6,211,205; 6,271,243; 6,288,095; 6,303,640; and 6,329,404; each of which is incorporated herein in its entirety. It is believed that the combined use of pantethine or a derivative thereof and a glitazone may increase glucose uptake in muscle and reduced endogenous glucose production.

Specific methods and pharmaceutical compositions of the invention comprise pantethine or a derivative thereof and 5-((4-(2-(methyl-2-pyridinyl amino)ethoxy)-phenyl)methyl)-2,4-thiazolidinedione or a pharmaceutically acceptable salt, solvate, clathrate, polymorph, prodrug, or pharmacologically active metabolite thereof.

Other specific methods and pharmaceutical compositions of the invention comprise pantethine or a derivative thereof and troglitazone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and pioglitazone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and ciglitazone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and WAY-120,744 or a pharmaceutically acceptable salt, solvate, clathrate, polymorph, prodrug, or pharmacologically active metabolite thereof.

Other specific methods and pharmaceutical compositions of the invention comprise pantethine or a derivative thereof and englitazone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and AD 5075 or a pharmaceutically acceptable salt, solvate, clathrate, polymorph, prodrug, or pharmacologically active metabolite thereof.

Other specific methods and pharmaceutical compositions of the invention comprise pantethine or a derivative thereof and darglitazone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and rosiglitazone or a pharmaceutically acceptable salt, solvate, clathrate, polymorph, prodrug, or pharmacologically active metabolite thereof.

### 4.2.5 Sulfonylureas

It is believed that a composition comprising pantethine or a derivative thereof and a sulfonylurea or a derivative thereof may increase insulin release from the pancreas and may further insulin levels by reducing hepatic clearance of the hormone.

Sulfonylurea-based drugs for use the compositions and methods of the invention include, but are not limited to, glisoxepid, glyburide, acetohexamide, chlorpropamide, glibomuride, tolbutamide, tolazamide, glipizide, gliclazide, gliquidone, glyhexamide, phenbutamide, tolcyclamide, combinations thereof, or a pharmaceutically acceptable salt, solvate, clathrate, polymorph, prodrug, or pharmacologically active metabolite thereof

Side effects of sulfonylureas include low blood sugar (hypoglycemia), weight gain, and allergic reactions in people with an allergy to sulfa medicines.

Specific methods and pharmaceutical compositions of the invention comprise pantethine or a derivative thereof and glisoxepid or a pharmaceutically acceptable salt, solvate, clathrate, polymorph, prodrug, or pharmacologically active metabolite thereof.

Other specific methods and pharmaceutical compositions of the invention comprise pantethine or a derivative thereof and glyburide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and acetohexamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and chlorpropamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and glibomuride or a pharmaceutically acceptable salt, solvate, clathrate, polymorph, prodrug, or pharmacologically active metabolite thereof.

Other specific methods and pharmaceutical compositions of the invention comprise pantethine or a derivative thereof and tolbutamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and tolazamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and glipizide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and gliclazide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and gliquidone or a pharmaceutically acceptable salt, solvate, clathrate, polymorph, prodrug, or pharmacologically active metabolite thereof.

Other specific methods and pharmaceutical compositions of the invention comprise pantethine or a derivative thereof and glyhexamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and phenbutamide or a pharmaceutically acceptable salt, solvate, clathrate, polymorph, prodrug, or pharmacologically active metabolite thereof.

Other specific methods and pharmaceutical compositions of the invention comprise pantethine or a derivative thereof and tolcyclamide or a pharmaceutically acceptable salt, solvate, clathrate, polymorph, prodrug, or pharmacologically active metabolite thereof

### 4.2.6 Dyslipidemic Controlling Compounds

Methods and compositions of the invention also can include the use of small dyslipidemic controlling compounds of the general formula: 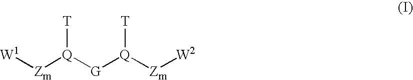
and pharmaceutically acceptable salts, solvates, prodrugs, enantiomers, diastereomers, geometric isomers, and mixtures thereof, wherein
(a) each occurrence of Z is independently CH₂, CH═CH, or phenyl, where each occurrence of m is independently an integer ranging from 1 to 9, but when Z is phenyl then its associated m is 1;
(b) G is —(CHOH), —S, —S(O), O, —C(O), or (CH₂)ₓ, where x is 2, 3, or 4, CH₂CH═CHCH₂, CH═CH, CH₂-phenyl-CH₂, or phenyl;
(c) W¹ and W² are independently L, V, C(R¹)(R²—CH₂)_{c}—C(R³)(R⁴l)(CH₂)ₙ—Y, or C(R¹)(R²)—(CH₂)_{c}—V where c is 1 or 2 and n is an integer ranging from 0 to 4;
(d) each occurrence of R¹ or R² is independently (C₁-C₆)alkyl, (C₂-C₆)alkenyl, (C₂-C₆)alkynyl, phenyl, or benzyl or when one or both of W¹ and W² is C(R¹)(R²)—(CH₂)_{c}—C(R³)(R⁴)—CH₂)ₙ—Y, then R¹ and R² can both be H to form a methylene group;
(e) R³ is H, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(f) R⁴ is OH, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(g) L is C(R¹)(R²)—CH₂)ₙ—Y;
(h) V is: 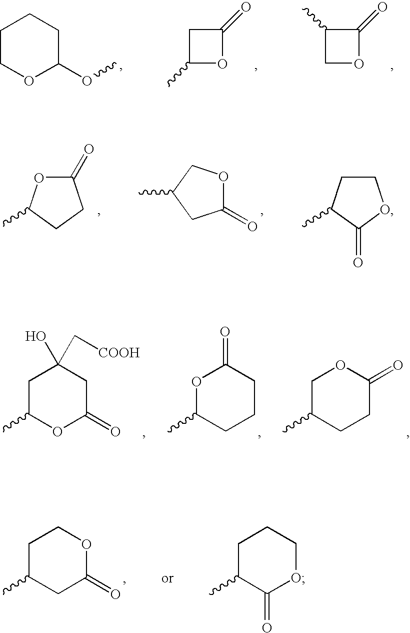
(i) each occurrence of Y is independently OH, COOH, CHO, COOR⁵, SO₃H, 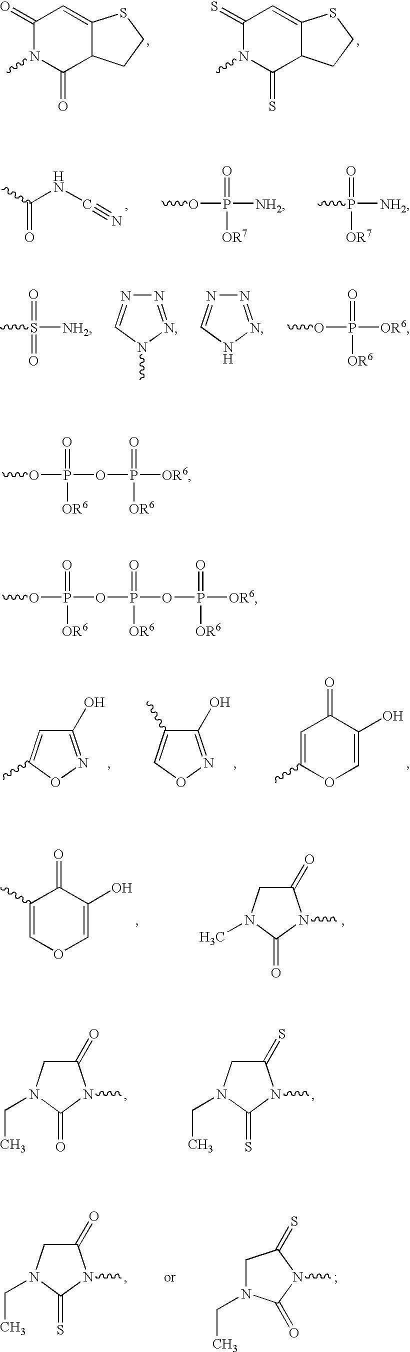
wherein:
   (i) R⁵ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
   (ii) each occurrence of R⁶ is independently H, (C₁-C₆)alkyl, (C₂-C₆)alkenyl, or (C₂-C₆)alkynyl and is unsubstituted or substituted with one or two halo, OH, (C₁-C₆) alkoxy, or phenyl groups; and
   (iii) each occurrence of R⁷ is independently H, (C₁-C₆)alkyl, (C₂-C₆)alkenyl, or (C₂-C₆)alkynyl;
(j) each occurrence of Q is independently C, CH, S, or O; and
(k) each occurrence of T is independently an electron pair, —H, —OH, or —(═O).

Dyslipidemic controlling compounds suitable for use in the compositions or methods of the invention are disclosed in U.S. application Ser. No. 09/976,899, filed Oct. 11, 2001; Ser. No. 09/976,867, filed Oct. 11, 2001; Ser. No. 09/976,898, filed Oct. 11, 2001; and Ser. No. 09/976,938, filed Oct. 11, 2001; each of which is incorporated herein in its entirety by reference.

In addition, the compositions and methods of the invention can also comprise pantethine, or a derivative thereof and sibutramine, or a pharmaceutically acceptable salt, solvate, clathrate, polymorph, prodrug, or pharmacologically active metabolite thereof. Sibutramine, chemically named [N-1-[1-(4-chlorophenyl)cyclobutyl]-3-methylbutyl]-N,N-dimethylamine. The invention encompasses racemic and stereomerically pure form of sibutramine. Sibutramine is disclosed in U.S. Pat. Nos. 4,552,828, 4,746,680, 4,806,570, 4,929,629, 4,871,774, 4,939,175, 5,436,272, each of which is incorporated herein in its entirety.

### 4.2.7 Small Peptides of the Invnetion

The peptides of the invention are generally capable of forming amphipathic α-helices in the presence of lipids as described in U.S. Pat. No. 6,004,925, the entire disclosure of which is incorporated herein by reference. Their main feature is a “core” peptide composed of 15 to 29 amino acid residues, preferably 22 amino acid residues, or an analogue thereof wherein at least one amide linkage in the peptide is replaced with a substituted amide, an isostere of an amide or an amide mimetic.

Peptides (or analogues thereof) of the invention have the following structural formula (II):
X₁—X₂—X₃—X₄—X₅—X₆—X₇—X₈—X₉—X₁₀—X₁₁—X₁₂—X₁₃—X₁₄—X₁₅—X₁₆—X₁₇—X₁₈—X₁₉—X₂₀—X₂₁—X₂₂ (II)
wherein:
X₁ is Pro (P), Ala (A), Gly (G), Gin (Q), Asn (N), Asp (D) or D-Pro (p);
X₂ is an aliphatic amino acid;
X₃ is Leu (L) or Phe (F);
X₄ is an acidic amino acid;
X₅ is Leu (L) or Phe (F);
X₆ is Leu (L) or Phe (F);
X₇ is a hydrophilic amino acid;
X₈ is an acidic or a basic amino acid;
X₉ is Leu (L) or Gly (G);
X₁₀ is Leu (L), Trp (W) or Gly (G);
X₁₁ is a hydrophilic amino acid;
X₁₂ is a hydrophilic acid;
X₁₃ is Gly (G) or an aliphatic amino acid;
X₁₄ is Leu (L), Trp (W), Gly (G) or Nal;
X₁₅ is a hydrophilic amino acid;
X₁₆ is a hydrophobic amino acid;
X₁₇ is a hydrophobic amino acid;
X₁₈ is a basic amino acid, Gln (Q) or Asn (N);
X₁₉ is a basic amino acid, Gln (Q) or Asn (N);
X₂₀ is a basic amino acid;
X₂₁ is an aliphatic amino acid; and
X₂₂ is a basic amino acid;
and pharmaceutically acceptable salts, solvates, clathrates, or prodrugs thereof.

In the peptides of structure (I), the symbol “—” between amino acid residues Xₙ generally designates a backbone constitutive linking function. Thus, the symbol “—” usually represents a peptide bond or amide linkage (—C(O)NH—). It is to be understood, however, that the present invention contemplates peptide analogues wherein one or more amide linkages is optionally replaced with a linkage other than amide, preferably a substituted amide or an isostere of amide. Thus, while the various Xₙ residues within structure (I) are generally described in terms of amino acids, and preferred embodiments of the invention are exemplified by way of peptides, one having skill in the art will recognize that in embodiments having non-amide linkages, the term “amino acid” or “residue” as used herein refers to other bifunctional moieties bearing groups similar in structure to the side chains of the amino acids.

Substituted amides generally include, but are not limited to, groups of the formula —C(O)NR—, where R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl and substituted 6-26 membered alkheteroaryl.

Isosteres of amide generally include, but are not limited to, —CH₂NH—, —CH₂S—, —CH₂CH₂—, —CH═CH— (cis and trans), —C(O)CH₂—, —CH(OH)CH₂— and —CH₂SO—. Compounds having such non-amide linkages and methods for preparing such compounds are well-known in the art (See, e.g., Spatola, March 1983, Vega Data Vol. 1, Issue 3; Spatola, 1983, “Peptide Backbone Modifications” In: Chemistry and Biochemistry of Amino Acids Peptides and Proteins, Weinstein, ed., Marcel Dekker, New York, p. 267 (general review); Morley, 1980, Trends Pharm. Sci. 1:463-468; Hudson et al., 1979, Int. J Prot. Res. 14:177-185 (—CH₂NH—, —CH₂CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA 97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—), each of which is incorporated herein.

Small peptides of the invention suitable for inclusion in the compositions or administration in the methods of the invention are disclosed in U.S. Pat. No. 6,004,925, which is incorporated herein by reference in its entirety.

### 4.3 Third Active Agents

In certain embodiments of the invention, one or more additional, or third active agents, is used in combination with pantethine, or a derivative thereof, and a second active agent. Preferred third active agents prevent or reduce the severity of an adverse effect associated with pantethine or the second active agent.

Specific third active agents are bile-acid-binding resins. Bile-acid-binding resins for use in combination with pantethine, or a derivative thereof, and a second active agent include, but are not limited to, cholestyramine and colestipol hydrochloride.

Additional third active agents are niacin or nicotinic acid.

Additional third active agents are RXR agonists. RXR agonists for use in combination with the compounds of the invention include but are not limited to LG 100268, LGD 1069, 9-cis retinoic acid, 2-(1-(3,5,5,8,8-pentamethyl-5,6,7,8-tetrahydro-2-naphthyl)-cyclopropyl)-pyridine-5-carboxylic acid, or 4-((3,5,5,8,8-pentamethyl-5,6,7,8-tetrahydro-2-naphthyl)2-carbonyl)-benzoic acid.

Additional third active agents are anti-obesity drugs. Anti-obesity drugs for use in combination with the compounds of the invention include but are not limited to β-adrenergic receptor agonists, preferably β-3 receptor agonists, fenfluramine, dexfenfluramine, sibutramine, bupropion, fluoxetine, and phentermine.

Additional third active agents are hormones. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

An additional third active agent is tyrophostine or analogs thereof. Tyrophostines for use in combination with the compounds of the invention include but are not limited to tryophostine 51.

Additional third active agents are α-glucosidase inhibitors. α-Glucosidase inhibitors for use in combination with the compounds of the invention include, but are not limited to, acarbose and miglitol.

Additional third active agents are apo A-I agonists. In one embodiment, the apo A-I agonist is the Milano form of apo A-I (apo A-IM). In a preferred mode of the embodiment, the apo A-IM for administration in conjunction with the compounds of the invention is produced by the method of U.S. Pat. No. 5,721,114 to Abrahamsen. In a more preferred embodiment, the apo A-I agonist is a peptide agonist. In a preferred mode of the embodiment, the apo A-I peptide agonist for administration in conjunction with the compounds of the invention is a peptide of U.S. Pat. Nos. 6,004,925 or 6,037,323 to Dasseux, each of which are incorporated herein by reference.

An additional third active agent is apolipoprotein E (apo E). In a preferred mode of the embodiment, the apoE for administration in conjunction with the compounds of the invention is produced by the method of U.S. Pat. No. 5,834,596 to Ageland, which is incorporated herein by reference.

Additional third active agents are HDL-raising drugs; HDL enhancers; or regulators of the apolipoprotein A-I, apolipoprotein A-IV and/or apolipoprotein genes.

Additional third active agents are anti-hypertensive agents. Anti-hypertensive agents for use in combination with the compounds of the invention include, but are not limited to, b-blockers, acteylcholinesterase (ACE) inhibitors, or angiotensin II receptor blockers. A specific anti-hypertensive agent is losartan.

Effective amounts of the third active agents are well known to those skilled in the art. However, it is well within the skilled artisan's purview to determine the third active agent's optimal effective-amount range.

### 4.3.1 Cardiovascular Drugs

Additional third active agents also include, but are not limited to, cardiovascular drugs. Cardiovascular drugs for use in combination with the compounds of the invention to prevent or treat cardiovascular diseases include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

### 4.3.2 Anti-Cancer Drugs

Additional third active agents include, but are not limited to, drugs administered together with treatment with irradiation or one or more chemotherapeutic agents. For irradiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2^{nd.} Ed., J. B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, additional third active agents further comprises one or more chemotherapeutic agents and/or is administered concurrently with radiation therapy.

### 4.4 Pharmaceutical Compositions and Single Unit Dosage Forms

Pharmaceutical compositions can be used in the preparation of individual, single unit dosage forms. Consequently, pharmaceutical compositions and dosage forms of the invention comprise the active ingredients disclosed herein (e.g., pantethine, or a derivative thereof, or one or more second active agents). Pharmaceutical compositions and dosage forms of the invention can further comprise one or more excipients.

Pharmaceutical compositions and dosage forms of the invention can also comprise one or more additional active ingredients. Examples of optional additional active ingredients are the “third active agents” disclosed herein (see, e.g., section 4.3).

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines (e.g., pantethine) are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical daily dosage forms of the invention comprise a pantethine or a derivative thereof in an amount of from about 1 to about 200 mg, from about 5 to about 100 mg, from about 10 to about 75 mg, or from about 20 to about 50 mg. Typical daily dosage forms comprise the second active agent or derivative thereof in an amount of from about 1 to about 1000 mg, from about 5 to about 500 mg, from about 10 to about 350 mg, or from about 50 to about 200 mg. The specific amount of second active agent will depend on the specific agent used, the type of disorder being treated, prevented, or managed, and the amount(s) of pantethine and any optional additional active agents concurrently administered to the patient.

In addition, the amounts and specific types of third active agents in a dosage form may differ depending on factors such as, but not limited to, the route by which it is to be administered to patients. These amounts will typically be known to those of ordinary skill in the art. See, generally, Physician's Desk Reference, 55^{th} ed., (2001), incoporated herein by reference.

### 4.4.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A specific solid oral dosage form of the invention comprises a small molecule-based active agent, anhydrous lactose, microcrystalline cellulose, polyvinylpyrrolidone, stearic acid, colloidal anhydrous silica, and gelatin.

### 4.4.2 Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of a small molecule-based active agent and its derivatives. See, e.g., U.S. Pat. No. 5,134,127, which is incorporated herein by reference.

### 4.4.4 Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 4.4.5 Kits

Typically, active ingredients of the invention are preferably not administered to a patient at the same time or by the same route of administration. This invention therefore encompasses kits containing for example instructions,which, when used by the medical practitioner, can simplify the administration of appropriate amounts of active ingredients to a patient.

A typical kit of the invention comprises a dosage form of a pantethine, or a derivative thereof, and a second active agent. Kits encompassed by this invention can further comprise additional active ingredients. Examples of optional additional active ingredients include, but are not limited to, those disclosed herein (see, e.g., section 4.3).

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 4.5 Therapeutic/Prophylactic Uses

This invention provides a method of treating, preventing, and managing a variety of diseases and conditions, which comprise adminstering an effective amount of pantethine, or a derivative thereof, and a second active agent to a patient in need of such treatment, prevention, or management. Where pantethine, or a derivative thereof, and a second active agent are administered to an animal, the effective amount of the pantethine is preferably less than what its effective amount would be if the second active agent were not administered. Similarly, the effective amount of the second active agent is preferably less than what its effective amount would be if the pantethine, or derivative thereof, were not administered. In such cases, without being bound by theory, it is believed that the pantethine, or derivative thereof, and the second active agent act synergistically to treat, prevent, or manage the cholesterol, dyslipidemia, or related disorder.

Examples of such diseases and disorders that can be treated, prevented, or managed by the methods of the invention include, but are not limited to, cholesterol, dyslipidemia, and related disorders such as, but not limited to, cardiovascular disease; artherosclerosis; stroke; peripheral vascular disease; dyslipidemia; dyslipoproteinemia; restenosis; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; impotence; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus (NIDDM); septic shock; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease.

In one embodiment, the terms “treat,” “treatment,” or “treating” refer to an amelioration of a disease or disorder, or at least one discernible symptom thereof. In another embodiment, the terms “treatment” or “treating” refer to an amelioration of at least one measurable physical parameter, not necessarily discernible by the patient. In yet another embodiment, the terms “treat,” “treatment,” or “treating” refer to inhibiting the progression of a disease or disorder, either physically, e.g., stabilization of a discernible symptom, physiologically, e.g., stabilization of a physical parameter, or both. In yet another embodiment, the terms“treat,” “treatment,” or “treating” refer to delaying the onset of a disease or disorder.

In certain embodiments, the compositions of the invention are administered to an animal, preferably a human, as a preventative measure against such diseases. As used herein, the terms“prevent,” “prevention,” or “preventing” refer to a reduction of the risk of acquiring a given disease or disorder. In a preferred mode of the embodiment, the compositions of the present invention are administered as a preventative measure to an animal, preferably a human, having a genetic predisposition to a cholesterol, dyslipidemia, or related disorders including, but not limited to, cardiovascular disease; artherosclerosis; stroke; peripheral vascular disease; dyslipidemia; dyslipoproteinemia; restenosis; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; impotence; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus (NIDDM); septic shock; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease. Examples of such genetic predispositions include but are not limited to the ε4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia.

In another method of the invention, the compounds of the invention or compositions of the invention are administered as a preventative measure to a patient having a non-genetic predisposition to a cholesterol, dyslipidemia, or related disorders. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease). Without being limited by theory it is believed that pantethine or a derivative thereof is effective when administered to a patient for more than thirty days. Accordingly, the invention encompasses methods of treating, preventing, or managing a cholesterol, dyslipidemia, or related disorder, which comprises administering for at least thirty days to a patient in need of such treatment, prevention, or management an effective amount of pantethine, or a derivative thereof, and a second active agent or a pharmaceutically acceptable salt, solvate, clathrate, polymorph, prodrug, or pharmacologically active metabolite thereof.

### 4.5.1 Cardiovascular Diseases

The invention provides methods for the treatment, prevention, or management of a cardiovascular disease. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysftnctions, in particular those dysfunctions affecting blood vessel elasticity; peripheral vascular disease; coronary heart disease; myocardial infarcation; cerebral infarction and restenosis.

### 4.5.2 Dyslipidemias

The invention provides methods for the treatment, prevention, or management of dyslipidemias.

As used herein, the term “dyslipidemias” refers to disorders that lead to or are manifested by aberrant levels of circulating lipids. To the extent that levels of lipids in the blood are too high, the methods of the invention can be used to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org and http://rover.nhlbi.nih.gov/chd/, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Dyslipidemias include, but are not limited to, hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of ketone bodies (e.g. β-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention further provides methods for altering lipid metabolism in an animal, e.g., reducing LDL in the blood of a patient, reducing free triglycerides in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

### 4.5.3 Dyslipoproteinemias

The invention provides methods for the treatment, prevention, or management of a dyslipoproteinemia comprising administering to an animal a composition comprising an effective amount of pantethine and a second active agent and a pharmaceutically acceptable vehicle.

As used herein, the term “dyslipoproteinemias” refers to disorders that lead to or are manifested by aberrant levels of circulating lipoproteins. To the extent that levels of lipoproteins in the blood are too high, the compositions of the invention are administered to a patient to restore normal levels. Conversely, to the extent that levels of lipoproteins in the blood are too low, the compositions of the invention are administered to a patient to restore normal levels. Normal levels of lipoproteins are reported in medical treatises known to those of skill in the art.

Dyslipoproteinemias include, but are not limited to, high blood levels of LDL; high blood levels of apolipoprotein B (apo B); high blood levels of Lp(a); high blood levels of apo(a); high blood levels of VLDL; low blood levels of HDL; reduced or deficient lipoprotein lipase levels or activity, including reductions or deficiencies resulting from lipoprotein lipase mutations; hypoalphalipoproteinemia; lipoprotein abnormalities associated with diabetes; lipoprotein abnormalities associated with obesity; lipoprotein abnormalities associated with Alzheimer's Disease; and familial combined hyperlipidemia.

The invention further provides methods for reducing apo C-II levels in the blood of an animal; reducing apo C-III levels in the blood of an animal; elevating the levels of HDL associated proteins, including but not limited to apo A-I, apo A-II, apo A-IV and apo E in the blood of a patient; elevating the levels of apo E in the blood of an animal, and promoting clearance of triglycerides from the blood of a patient, said methods comprising administering to the patient a compound or a composition comprising a compound of the invention in an amount effective to bring about said reduction, elevation or promotion, respectively.

### 4.5.4 Glucose Metabolism Disorders

The invention provides methods for the treatment, prevention, or management of a glucose metabolism disorder. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the methods of the invention can restore normal levels. Normal indicia of glucose metabolism are reported in medical treatises known to those of skill in the art.

Glucose metabolism disorders which the methods of the invention are useful for preventing, treating, or managine include, but are not limited to, impaired glucose tolerance; diabetic retinopathy, diabetic nephropathy, insulin resistance; insulin resistance related breast, colon or prostate cancer; diabetes, including but not limited to non-insulin dependent diabetes mellitus (NIDDM), insulin dependent diabetes mellitus (IDDM), gestational diabetes mellitus (GDM), and maturity onset diabetes of the young (MODY); pancreatitis; hypertension; polycystic ovarian disease; and high levels of blood insulin and/or glucose.

The present invention further provides methods for altering glucose metabolism in a patient, for example to increase insulin sensitivity and/or oxygen consumption of an animal, said methods comprising administering to an animal a composition comprising pantethine and a second active agent in an amount effective to alter glucose metabolism.

### 4.5.5 PPAR Associated Disorders

The invention provides methods for the treatment, prevention, or management of PPAR-associated disorders. As used herein, “treatment, prevention, or managment of PPAR associated disorders” encompasses treatment, prevention, or management of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL may be elevated in lymph and/or cerebral fluid.

### 4.5.6 Renal Diseases

The invention provides methods for the treatment, prevention, or management of renal diseases. Renal diseases that can be treated, prevented, or managed by the methods of the invention include, but are not limited to, glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal disease, medullary sponge kidney, medullary cystic disease, nephrogenic diabetes, and renal tubular acidosis), tubulointerstitial diseases (including but not limited to pyelonephritis, drug and toxin induced tubulointerstitial nephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

### 4.5.7 Cancers

The invention provides methods for the treatment, prevention, or management of cancer. Cancers that can be treated, prevented, or managed by the methods of the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. In a most preferred embodiment, cancers that are treated or prevented by administering the compounds of the present invention are insulin resistance or Syndrome X related cancers, including but not limited to breast, prostate and colon cancer.

### 4.5.8 Other Diseases

The invention provides methods for the treatment, prevention, or management of neurodegenerative disease or disorders, Parkinson's Disease, Alzheimer's Disease, Syndrome X, septicemia, thrombotic disorders, obesity, pancreatitis, hypertension, inflammation, and impotence.

As used herein, “treatment, prevention, or management of Alzheimer's Disease” encompasses treatment, prevention, or management of lipoprotein abnormalities associated with Alzheimer's Disease.

As used herein, “treatment, prevention, or management of Syndrome X or Metabolic Syndrome” encompasses treatment, prevention, or management of a symptom thereof, including but not limited to impaired glucose tolerance, hypertension and dyslipidemia/dyslipoproteinemia.

As used herein, “treatment, prevention, or management of septicemia” encompasses treatment, prevention, or management of septic shock.

As used herein, “treatment, prevention, or management of thrombotic disorders” encompasses treatment, prevention, or management of high blood levels of fibrinogen and promotion of fibrinolysis.

In addition to treatment, prevention, or management in obesity, the compositions of the invention can be administered to an individual to promote weight reduction of the individual.

### 4.6 Surgical Uses of the Compounds and Compositions of the Invention

Cardiovascular diseases such as atherosclerosis often require surgical procedures such as angioplasty. Angioplasty is often accompanied by the placement of a reinforcing a metallic tube-shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compositions of the invention may be used as coatings on surgical devices (e.g., catheters) and implants (e.g., stents) to reduce the risk of restenosis and thrombosis associated with invasive procedures used in the treatment of cardiovascular diseases.

### 4.7 Veterinary and Livestock Uses of the Compounds and Compositions of the Invention

A composition of the invention can be administered to a non-human animal for a veterinary use for treating, preventing, or managing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

In addition to veterinary uses, the compounds and compositions of the invention can be used to reduce the fat content of livestock to produce leaner meats. Alternatively, the compounds and compositions of the invention can be used to reduce the cholesterol content of eggs by administering the compounds to a chicken, quail, or duck hen. For non-human animal uses, the compounds and compositions of the invention can be administered via the animals' feed or orally as a drench composition.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which have been incorporated herein by reference in their entirety.

1. A pharmaceutical composition comprising pantethine or a derivative thereof and a second active agent, wherein the second active agent is a statin, fibrate, biguanide, glitazone, sulfonylurea, dyslipidemic controlling compound, or a peptide.

2. The pharmaceutical composition of claim 1, wherein the second active agent is a statin, or a pharmaceutically acceptable salt, solvate, clathrate, polymorph, prodrug, or pharmacologically active metabolite thereof.

3. The pharmaceutical composition of claim 1, wherein the second active agent is a fibrate, or a pharmaceutically acceptable salt, solvate, clathrate, polymorph, prodrug, or pharmacologically active metabolite thereof.

4. The pharmaceutical composition of claim 1, wherein the second active agent is a biguanide, or a pharmaceutically acceptable salt, solvate, clathrate, polymorph, prodrug, or pharmacologically active metabolite thereof.

5. The pharmaceutical composition of claim 1, wherein the second active agent is a glitazone, or a pharmaceutically acceptable salt, solvate, clathrate, polymorph, prodrug, or pharmacologically active metabolite thereof.

6. The pharmaceutical composition of claim 1, wherein the second active agent is a sulfonylurea, or a pharmaceutically acceptable salt, solvate, clathrate, polymorph, prodrug, or pharmacologically active metabolite thereof.

7. The pharmaceutical composition of claim 1, wherein the second active agent is a dyslipidemic controlling compound of formula: 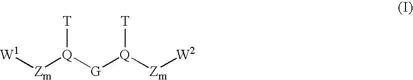
or a pharmaceutically acceptable salt, solvate, clathrate, polymorph, prodrug, pharmacologically active metabolite, enantiomer, diastereomer, geometric isomer or mixtures thereof, wherein
(a) each occurrence of Z is independently CH₂, CH═CH, or phenyl, where each occurrence of m is independently an integer ranging from 1 to 9, but when Z is phenyl then its associated m is 1;
(b) G is —(CHOH), —S, —S(O), O, or (CH₂)ₓ, where x is 2, 3, or 4, CH₂CH═CHCH₂, CH═CH, CH₂-phenyl-CH₂, or phenyl;
(c) W¹ and W² are independently L, V, C(R¹)(R²)—CH₂)_{c}—C(R³)(R⁴)—CH₂)ₙ—Y, or C(R¹)(R²)—CH₂)_{c}—V where c is 1 or 2 and n is an integer ranging from 0 to 4;
(d) each occurrence of R¹ or R² is independently (C₁-C₆)alkyl, (C₂-C₆)alkenyl, (C₂-C₆)alkynyl, phenyl, or benzyl or when one or both of W¹ and W² is C(R¹)(R²)—(CH₂)_{c}—C(R³)(R⁴)—CH₂)ₙ—Y, then R¹ and R² can both be H to form a methylene group;
(e) R³ is H, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(f) R⁴ is OH, (C₁-C₆)alkyl, (C₂-C₆)alkenyl, (C₂-C₆)alkynyl, (C₁-C₆)alkoxy, phenyl, benzyl, Cl, Br, CN, NO₂, or CF₃;
(g) L is C(R¹)(R²)—CH₂)ₙ—Y;
(h) V is: 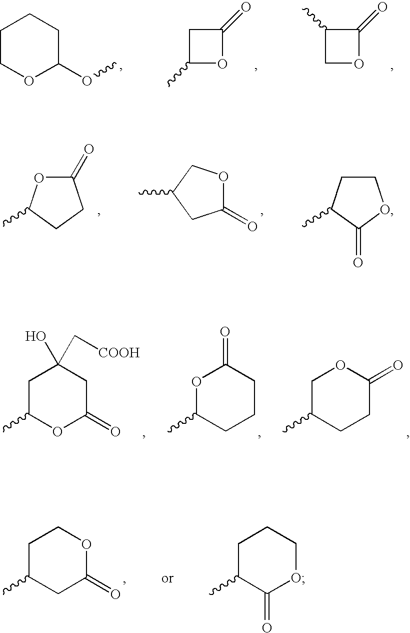
(i) each occurrence of Y is independently OH, COOH, CHO, COOR⁵, SO₃H, 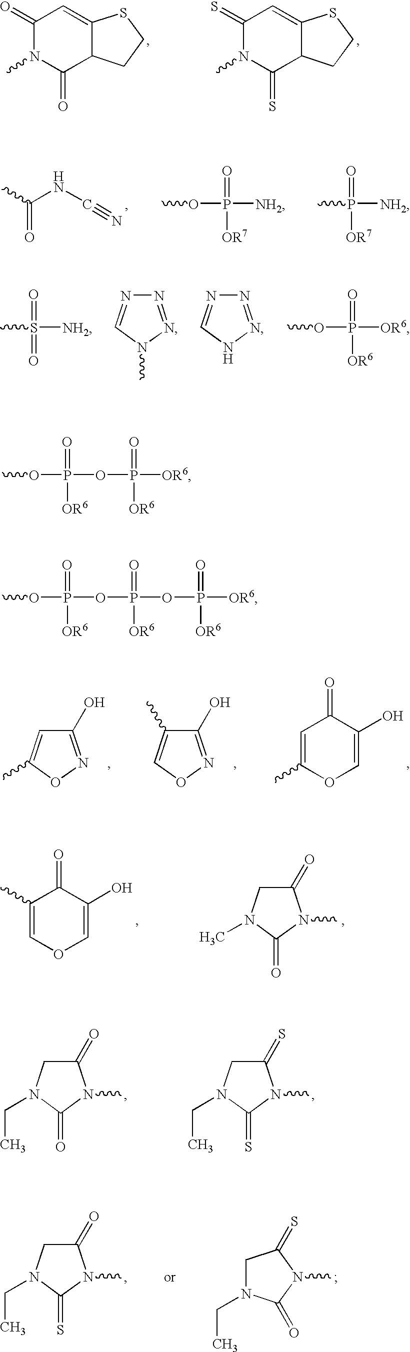
wherein:
(i) R⁵ is (C₁-C₆)alkyl, (C₂-C₆)alkenyl, (C₂-C₆)alkynyl, phenyl, or benzyl and is unsubstituted or substituted with one or more halo, OH, (C₁-C₆)alkoxy, or phenyl groups,
(ii) each occurrence of R⁶ is independently H, (C₁-C₆)alkyl, (C₂-C₆)alkenyl, or (C₂-C₆)alkynyl and is unsubstituted or substituted with one or two halo, OH, (C₁-C₆) alkoxy, or phenyl groups; and
(iii) each occurrence of R⁷ is independently H, (C₁-C₆)alkyl, (C₂-C₆)alkenyl, or (C₂-C₆)alkynyl;
(j) each occurrence of Q is independently C, CH, S, or O; and
(k) each occurrence of T is independently an electron pair, —H, —OH, or —(═O);
or a pharmaceutically acceptable salt, solvate, prodrug, or combination thereof.

8. The pharmaceutical composition of claim 1, wherein the second active agent is a peptide of formula (II):
X₁—X₂—X₃—X₄—X₅—X₆—X₇—X₈—X₉—X₁₀—X₁₁—X₁₂—X₁₃—X₁₄—X₁₅—X₁₆—X₁₇—X₁₈—X₁₉—X₂₀—X₂₁—X₂₂ (II)
wherein:
X₁ is Pro (P), Ala (A), Gly (G), Gln (Q), Asn (N), Asp (D) or D-Pro (p);
X₂ is an aliphatic amino acid;
X₃ is Leu (L) or Phe (F);
X₄ is an acidic amino acid;
X₅ is Leu (L) or Phe (F);
X₆ is Leu (L) or Phe (F);
X₇ is a hydrophilic amino acid;
X₈ is an acidic or a basic amino acid;
X₉ is Leu (L) or Gly (G);
X₁₀ is Leu (L), Trp (W) or Gly (G);
X₁₁ is a hydrophilic amino acid;
X₁₂ is a hydrophilic acid;
X₁₃ is Gly (G) or an aliphatic amino acid;
X₁₄ is Leu (L), Trp (W), Gly (G) or Nal;
X₁₅ is a hydrophilic amino acid;
X₁₆ is a hydrophobic amino acid;
X₁₇ is a hydrophobic amino acid;
X₁₈ is a basic amino acid, Gln (Q) or Asn (N);
X₁₉ is a basic amino acid, Gln (Q) or Asn (N);
X₂₀ is a basic amino acid;
X₂₁ is an aliphatic amino acid; and
X₂₂ is a basic amino acid;
or a pharmaceutically acceptable salt, solvate, clathrate, polymorph, prodrug, or pharmacologically active metabolite thereof.

9. The pharmaceutical composition of claim 1, further comprising a pharmaceutically acceptable carrier or excipient.

10. The pharmaceutical composition of claim 2, wherein the statin is mevastatin, lovastatin, simvastatin, pravastatin, fluvastatin, pitavastatin, atorvastatin, cerivastatin, or a pharmaceutically acceptable salt, solvate, clathrate, polymorph, prodrug, or pharmacologically active metabolite thereof.

11. The pharmaceutical composition of claim 3, wherein the fibrate is benzafibrate, ciprofibrate, fenofibrate, gemfibrozil, clofibrate, or a pharmaceutically acceptable salt, solvate, clathrate, polymorph, prodrug, or pharmacologically active metabolite thereof.

12. The pharmaceutical composition of claim 5, wherein the glitazone is troglitazone, rosiglitazone, pioglitazone, or a pharmaceutically acceptable salt, solvate, clathrate, polymorph, prodrug, or pharmacologically active metabolite thereof.

13. The pharmaceutical composition of claim 6, wherein the sulfonylurea is tolbutamide, chlorpropamide, tolazamide, acetohexamide, glyburide, glibenclamide, glipizide, gliclazide, glimepiride, or a pharmaceutically acceptable salt, solvate, clathrate, polymorph, prodrug, or pharmacologically active metabolite thereof.

14. The pharmaceutical composition of claim 1, further comprising a third active agent, or a pharmaceutically acceptable salt, solvate, clathrate, polymorph, prodrug, or pharmacologically active metabolite thereof.

15. The pharmaceutical composition of claim 14, wherein the third active agent is a bile acid-binding resin, niacin, RXR agonist, anti-obesity drug, hormone, tyrophostine, tyrophostine analogue, a-glucosidase inhibitor, apo A-1 agonist, apolipoprotein E, or a pharmaceutically acceptable salt, solvate, clathrate, polymorph, prodrug, or pharmacologically active metabolite thereof.

16. The pharmaceutical composition of claim 1, wherein the pharmaceutical composition is in tablet form.

17. The pharmaceutical composition of claim 1, wherein the pharmaceutical composition is in capsule form.

18. The pharmaceutical composition according to claim 1, wherein the pharmaceutical composition is in controlled released form.

19-65. (canceled)

66. A pharmaceutical composition comprising D,D-pantethine or pharmaceutically acceptable salt, solvate, clathrate, polymorph, or prodrug thereof and a second active agent.

67. A pharmaceutical composition comprising L,L-pantethine or pharmaceutically acceptable salt, solvate, clathrate, polymorph, or prodrug thereof and a second active agent.

68. A pharmaceutical composition comprising D,L-pantethine or pharmaceutically acceptable salt, solvate, clathrate, polymorph, or prodrug thereof and a second active agent.

69. A pharmaceutical composition comprising L,D-pantethine or pharmaceutically acceptable salt, solvate, clathrate, polymorph, or prodrug thereof and a second active agent.
